(19) United States
(12) Patent Application Publication
(10) Pub. No.: US 2003/0017491 A1
(43) Pub. Date: Jan. 23, 2003

(54) Chromogenic in situ hybridization methods, kits, and compositions

(76) Inventors: Zuo-Rong Shi, Redwood City, CA (US); Rina Wu, San Francisco, CA (US)

Correspondence Address: Jason R. Bond, MEDLEN & CARROLL, LLP, Suite 350, 101 Howard Street, San Francisco, CA, 94105, US

(21) Appl. No.: 10/173,525

(22) Filed: Jun. 17, 2002

Related U.S. Application Data
(63) Continuation-in-part of application No. 09/952,851, filed on Sep. 14, 2001.
(60) Provisional application No. 60/232,660, filed on Sep. 14, 2000.

(51) Int. Cl. C12Q 1/68; C12P 19/34

(52) U.S. Cl. 435/6; 435/91.2

(57) ABSTRACT

The present invention relates to chromogenic (colorimetric) in situ hybridization (CISH) and nucleic acid probes useful for in situ hybridization. Specifically, the present invention provides methods, kits, and compositions for performing bright field cancer diagnostics employing chromogenic in situ hybridization (e.g. to detect gene amplifications, gene translocations, and chromosome polysomy). In preferred embodiments, the present invention provides CISH methods, kits and compositions for detecting HER2 gene status.

The present application is a continuation-in-part of U.S. application Ser. No. 09/952,851, filed Sep. 14, 2001, which claims priority to U.S. Provisional Application Serial No. 60/232,660, filed Sep. 14, 2000, both of which are herein incorporated by reference.

### FIELD OF THE INVENTION

The present invention relates to chromogenic (calorimetric) in situ hybridization (CISH) and nucleic acid probes useful for in situ hybridization. Specifically, the present invention provides methods, kits, and compositions for performing bright-field cancer diagnostics employing chromogenic in situ hybridization (e.g. to detect gene amplifications, gene translocations, deletion, and chromosome aneuploidy). In preferred embodiments, the present invention provides CISH methods, kits and compositions for detecting HER2 (erbB-2) gene status.

### BACKGROUND OF THE INVENTION

Characterization chromosome aberrations have been studied in a wide range of tumors. Specific oncogene and tumor suppressor gene targets affected by these chromosomal abnormalities have been characterized in many tumors. One such target is the HER2 gene. HER2 gene amplification or HER2 protein overexpression has been identified in 10-34% of invasive breast cancers according to a series of 52 published studies including more than 16,000 patients and using different methodologies (See, Ross et al., Am. J. Clin. Pathol., 1999; 112:S53-67, herein incorporated by reference).

Identification of HER2 status is important for determining the prognosis of patients who have invasive breast cancer, as well as for selecting a subgroup with metastasis HER2 overexpression for therapy with trastuzumab (HERCEPTIN), a humanized anti-HER2 monoclonal antibody (See, Shak et al., Cancer Res. 199; 6:71-7; and Cobleigh et al., J. Clin. Oncol., 1999; 17:2639--48, both of which are herein incorporated by reference). HERCEPTIN has been found to be effective only in patients whose tumors show HER2 gene amplification and/or HER protein overexpression. As such, accurate, consistent, and straightforward methods for evaluation of HER2 status have become increasingly important.

Immunohistochemical (IHC) staining has been the predominant method of determining HER2 status in breast cancer specimens. It is relatively easy to perform and has a rapid turnaround time, and a relatively low cost (See, Ross et al. above, and Hanna et al., Mod. Pathol., 1999, 12:827-34, herein incorporated by reference). However, many commercially available antibodies have demonstrated wide variation in sensitivity and specificity for FFPE (formalin fixed paraffin embedded) tissue samples, and the effect of the tissue fixative and pretreament have a substantial effect on HER IHC staining (See, Ross et al. above; Jacobs et al., J. Clin. Oncol. 1999, 17:1974-1987; Espinoza et al., J. Clin. Oncol. 1999, 17:2293B; and Penault-Llorca et al., J. Pathol. 1994, 173:65-75, all of which are herein incorporated by reference). In addition, the lack of a universal scoring system and interobserver differences in interpretation of HER2 IHC results is also source of unwanted variation.

Overexpresion of the HER2 protein generally (>95%) results from HER2 gene amplification (See, Slamon et al., Science, 1989; 244:707-12, herein incorporated by reference). Fluorescence in situ hybridization (FISH) is believed by many to be the most sensitive technique for quantitative evaluation of HER2 gene status in breast cancer cells and also believed to be a valid alternative to IHC in FFPE tissue sections (See, Pauletti et al., J. Clin. Oncology, 2000, 18:3651-64, herein incorporated by reference.). Patients who were positive by FISH but negative by IHC had a worse survival rate than those who had HER2 overexpression but an absence of gene amplification (See, Pauletti et al., above). Therefore, HER2 amplification could provide more meaningful prognostic information than HER2 overexpression in breast cancer patients. In addition, FISH quantifies the number of gene copies in the cancer cell, which objectively reflects the HER2 gene status of tumors, whereas IHC is a more subjective test. Therefore, FISH can be easier to interpret than IHC. However, FISH methodology also has many disadvantages.

Evaluation of FISH requires a modern and expensive fluorescence microscope equipped with high-quality 60× or 100× oil immersion objectives and multi-band-pass fluorescence filters, which is not used in most routine diagnostic laboratories. The fluorescence signals can fade within several weeks, and the hybridization results are typically recorded with an expensive CCD camera. Therefore, analysis and recording of FISH data is expensive and time consuming. Most importantly, tissue section morphology is not optimal in FISH on FFPE, a particular problem for distinguishing invasive breast cancer and breast carcinoma in situ, where HER2 gene amplification or protein overexpression may have different clinical significance. All of these limitations make FFPE FISH cumbersome for routine work (See, Jacobs et al. above, and Tanner et al., Am. J. Pathol. 2000, 157:1467-72, herein incorporated by reference).

Therefore, what is needed are methods, kits and compositions that accurately identify cancer marker gene status, such as HER2 gene status, that do not require expensive fluorescence detection equipment, allow cell morphology and ISH signal to be viewed at the same time, and provide accurate results using standard equipment, such as bright field-microscopes.

### SUMMARY OF THE INVENTION

The present invention relates to chromogenic (colorimetric) in situ hybridization (CISH) and nucleic acid probes useful for in situ hybridization. Specifically, the present invention provides methods, kits, and compositions for performing bright-field cancer diagnostics employing chromogenic in situ hybridization (e.g. to detect gene amplifications, gene translocations, and chromosome polysomy). In preferred embodiments, the present invention provides CISH methods, kits and compositions for detecting HER2 gene status.

In some embodiments, the present invention provides methods for performing chromogenic in-situ hybridization, comprising; a) providing; i) a biological sample (e.g. tumor biopsy), ii) a labeled subtracted probe library, wherein the subtracted probe library is configured to hybridize to a target region, iii) pretreatment buffer, iv) enzyme digestion solution, v) a calorimetric substrate, and vi) a detection molecule conjugated to a calorimetric substrate enzyme; b) preheating the biological sample in the pretreatment buffer at a temperature of at least 96 degrees Celsius, c) exposing the biological sample to the enzyme digestion solution, d) contacting the biological sample with the subtracted probe library under conditions such that the subtracted probe library hybridizes to the target region, e) adding the detection molecule to the biological sample under conditions such that the detection molecule binds; i) to the labeled subtracted probe library, or ii) an intermediate molecule linked to the subtracted probe library, f) adding the colorimetric substrate to the biological sample under conditions such that the subtracted probe library is detected.

In particular embodiments, the present invention provides methods for performing chromogenic in-situ hybridization, comprising; a) preheating a biological sample (e.g. tumor biopsy) in a pretreatment buffer at a temperature of at least 96 degrees Celsius, b) exposing the biological sample to a enzyme digestion solution, c) contacting the biological sample with a subtracted probe library under conditions such that the subtracted probe library hybridizes to a target region in the biological sample, d) adding a detection molecule linked to an enzyme to the biological sample under conditions such that the detection molecule binds; i) to the labeled subtracted probe library, or ii) an intermediate molecule linked to the subtracted probe library, and e) adding a colorimetric substrate to the biological sample. In other embodiments, the method further comprises step f) detecting the presence or absence of the target region in the biological sample. In additional embodiments, the detecting comprising visualizing the calorimetric substrate with a microscope (e.g. bright-field microscope).

In some embodiments, the subtracted probe library is configured for detecting HER2 gene amplification. In particular embodiments, the target region comprises the HER2 gene. In other embodiments, the subtracted probe library is configured for detecting topolla gene amplification. In certain embodiments, the target region comprises the topolla gene (e.g. and does not encompass the HER2 gene sequence). In some embodiments, the subtracted probe library is configured for detecting EGFR (epidermal growth factor receptor) gene amplification. In particular embodiments, the target region comprises the EGFR gene. In other embodiments, the subtracted probe library is configured for detecting N-MYC gene amplification. In additional embodiments, the target region comprises the N-MYC gene.

In some embodiments, the subtracted probe library comprises a probe pair library. In other embodiments, the probe pair comprises a split-apart probe pair. In particular embodiments, the probe pair library comprises; i) a first probe library configured to hybridize to a first region of chromosome nine that is centromeric with respect to the ABL gene, and ii) a second probe library configured to hybridize to a second region of chromosome nine that is teleomeric with respect to the ABL gene. In other embodiments, the probe pair library comprises; i) a first probe library configured to hybridize to a first region of chromosome eighteen that is centromeric with respect to the SYT gene, and ii) a second probe library configured to hybridize to a second region of chromosome eighteen that is teleomeric with respect to the SYT gene.

In certain embodiments, the preheat temperature is at least 98 degrees Celsius (e.g. 98, 99 or 100 degrees Celsius). In other embodiments, the preheat temperature is from 96 degrees Celsius to 100 degrees Celsius (e.g. 98-100 degrees Celsius). In some embodiments, the preheating is accomplished with a pressure cooker, a hot plate, or a microwave oven. In other embodiments, the biological sample, during the preheating step, is inside an enclosed container.

In some embodiments, the enzyme digestion solution comprises pepsin (e.g., a solution having about 0.0625% pepsin, pH 2.3). In other embodiments, the pretreatment buffer comprises TRIS-EDTA (e.g. 0.1 M Tris/0.05 EDTA, pH 7.0). In other embodiments, the pretreament buffer is TRIS.

In certain embodiments, the detection molecule is avidin, streptavidin or biotin. In some embodiments, the detection molecule is an antibody. In particular embodiments, the detection molecule is linked to a plurality of enzymes via a polymer. In additional embodiments, the intermediate molecule is a primary antibody, and the detection molecule is a secondary antibody that binds to the primary antibody.

In some embodiments, the enzyme comprises a peroxidase (e.g. a horseradish peroxidase). In other embodiments, the enzyme is HRP or AP. In other embodiments, the method further comprises performing immunohistochemistry on the biological sample with antibodies specific for proteins expressed by the target region. In some embodiments, the subtracted probe library comprises digoxigenin, FITC, avidin, streptavidin, or biotin. In additional embodiments, the calorimetric substrate comprises diaminobenzidine or FAST RED.

In certain embodiments, the subtracted probe library comprises a heterogeneous mixture of labeled nucleic acid probes about 0.1 kb to about 8 kb in length (e.g. about 0.5 to about 4 kb in length). In some embodiments, the target region is about 50 kb to about 500 kb, or 1.5 to 5.0 megabases in length. In other embodiments, the target region is associated with human cancer gene aberrations. In certain embodiments, the biological sample is a tumor sample (e.g. a breast cancer biopsy tissue sample). In some embodiments, the biological sample is fixed on a surface (e.g. microscope slide).

In some embodiments, the subtracted probe library is about 90 percent free of repeat sequences. In other embodiments, the subtracted probe library is about 95 percent free of repeat sequences. In certain embodiments, the biological sample is a paraffin-embedded tissue sample (e.g. formalin-fixed paraffin-embedded tissue sample).

In particular embodiments, the preset invention provides kits for performing chromogenic in-situ hybridization, comprising; a) a labeled subtracted probe library, wherein the subtracted probe library is configured to hybridize to a target region, b) a written insert component, wherein the written inert component comprises instructions for performing chromogenic in-situ hybridization. In other embodiments, the kit further comprises at least one of the following; a pretreatment buffer, an enzyme digestion solution, a colorimetric substrate, and a detection molecule conjugated to a colorimetric substrate enzyme.

In additional embodiments, the instructions for performing chromogenic in-situ hybridization comprises instructions for visualizing the colorimetric substrate with a bright-field microscope. In certain embodiments, the subtracted probe library is configured for detecting HER2 gene amplification, topolla gene amplification, EGFR gene amplification, or N-MYC gene amplification.

In some embodiments, the subtracted probe library comprises a probe pair library. In other embodiments, the probe pair comprises a split-apart probe pair. In particular embodiments, the probe pair library comprises; i) a first probe library configured to hybridize to a first region of chromosome nine that is centromeric with respect to the ABL gene, and ii) a second probe library configured to hybridize to a second region of chromosome nine that is teleomeric with respect to the ABL gene. In other embodiments, the probe pair library comprises; i) a first probe library configured to hybridize to a first region of chromosome eighteen that is centromeric with respect to the SYT gene, and ii) a second probe library configured to hybridize to a second region of chromosome eighteen that is teleomeric with respect to the SYT gene.

In other embodiments, the written insert component comprises instructions for preheating a biological sample in a pretreament buffer to a temperature of at least 96 degrees Celsius. In some embodiments, the written insert component comprises instructions for preheating a biological sample in a pretreament buffer to a temperature of at least 98 degrees Celsius (e.g. 98-100 degrees Celsius). In certain embodiments, the instructions for preheating indicate that the temperature is accomplished with a pressure cooker, a hot plate or a microwave oven. In particular embodiments, the instructions for preheating further indicate that the biological sample, during the preheating step, should be inside an enclosed container.

In some embodiments, the present invention provides methods for diagnosing and treating a subject, comprising; a) preheating a biological sample from a subject in a pretreatment buffer, b) exposing the biological sample to a enzyme digestion solution, c) contacting the biological sample with a subtracted probe library under conditions such that the subtracted probe library hybridizes to a target region in the biological sample, wherein the target region comprises the HER2 gene sequence, d) adding a detection molecule linked to an enzyme to the biological sample under conditions such that the detection molecule binds; i) to the labeled subtracted probe library, or ii) an intermediate molecule linked to the subtracted probe library, e) adding a colorimetric substrate to the biological sample, f) detecting the target region by visualizing the colorimetric substrate with a bright-field microscope, thereby determining that the biological sample has amplification of the HER2 gene sequence, and g) identifying the subject as suitable for treatment with anti-HER2 antibodies. In particular embodiments, the method further comprises step h) administering the anti-HER2 antibodies (e.g. HERCEPTIN) to the subject.

In some embodiments, the present invention provides methods for identifying suitable treatment for a subject, comprising: screening a biological sample for the presence or absence of gene amplification in both HER-2/neu and topolla, wherein the biological sample is suspected of containing breast cancer cells and is obtained from the subject.

In other embodiments, the present invention provides methods for identifying suitable treatment for a subject, comprising: a) screening a biological sample for the presence or absence of: i) gene amplification in topolla and ii) gene amplification in HER-2/neu or overexpression of HER2, wherein the biological sample is suspected of containing breast cancer cells and is obtained from the subject, and b) identifying the subject as suitable for; i) anti-HER2 antibody-free anthracycline treatment, or ii) anthracycline-free anti-HER2 antibody treatment.

In some embodiments, the identifying the subject as suitable for anti-HER2 antibody-free anthracycline treatment comprises determining the presence of gene amplification in both said HER-2/neu and said topolla, or determining the presence of gene amplification in said topolla gene and overexpression of HER2. In other embodiments, the identifying the subject as suitable for anthracyline-free anti-HER2 antibody treatment comprises determining: i) the presence of gene amplification in the HER-2/neu or overexpression of HER2, and ii) the absence of gene amplification in the topolla.

In certain embodiments, the determining comprises performing in-situ hybridization methods on the biological sample with HER-2/neu and topolla specific probes. In additional embodiments, the in-situ hybridization methods comprise fluorescent in situ hybridization and/or chromogenic in situ hybridization. In other embodiments, the determining comprises performing in situ hybridization on the biological sample with a topolla specific probe, and performing immunohistochemical methods on the biological sample with anti-HER2 antibodies. In some embodiments, the methods further comprise step c) administering an anthracycline to the subject without administering anti-HER2 antibodies.

In certain embodiments, the identifying the subject as suitable for anthracyline-free anti-HER2 antibody treatment comprises determining: i) the presence of gene amplification in the HER-2/neu or overexpression of HER2, and ii) the absence of gene amplification in the topolla. In certain embodiments, the determining comprises performing in situ hybridization methods on the biological sample with HER-2/neu and topolla specific probes. In additional embodiments, the in situ hybridization methods comprise fluorescent in-situ hybridization and/or chromogenic in situ hybridization. In other embodiments, the determining comprises performing in situ hybridization on the biological sample with a topolla specific probe, and performing immunohistochemical methods on the biological sample with anti-HER2 antibodies. In particular embodiments, the methods further comprise step c) administering anti-HER2 antibodies (e.g. HERCEPTIN) to the subject without administering an anthracycline.

In some embodiments, the present invention provides kits for identifying suitable treatment for a subject, comprising: a) reagents for screening a biological sample from a subject, suspected of containing breast cancer cells, for the presence or absence of; i) gene amplification in topolla, and ii) gene amplification in HER-2/neu or HER overexpression, and b) a written insert component, wherein the written insert component comprises instructions for employing the reagents for identifying the subject as suitable for; i) anti-HER2 antibody-free anthracycline treatment, or ii) anthracycline-free anti-HER2 antibody treatment. In particular embodiments, the instructions for identifying the subject as suitable for anti-HER2 antibody-free anthracycline treatment comprises instructions for determining the presence of gene amplification in both the HER-2/neu and the topolla, or determining the presence of topolla gene amplification and HER-2/neu amplification or HER2 overexpression, employing the reagents.

In additional embodiments, the instructions for determining comprises instructions for performing in-situ hybridization methods (e.g., FISH and/or CISH) on the biological sample with HER-2/neu and topolla specific probes. In some embodiments, the instructions for determining comprises instructions for performing in-situ hybridization on the biological sample with a topolla specific probe, and instructions for performing immunohistochemical methods on the biological sample with anti-HER2 antibodies.

In other embodiments, the reagents comprise at least one of the following: a labeled subtracted probe library, wherein the subtracted probe library is configured to hybridize to a HER-2/neu or topolla, pretreatment buffer, an enzyme digestion solution, a calorimetric substrate, and a detection molecule conjugated to a colorimetric substrate enzyme. In some embodiments, the instructions for identifying the subject as suitable for anthracyline-free anti-HER2 antibody treatment comprises instructions for determining: i) the presence of gene amplification in the HER-2/neu or HER2 overexpression, and ii) the absence of gene amplification in the topolla. In certain embodiments, the instructions for determining comprises instructions for performing in-situ hybridization methods (e.g. FISH and/or CISH) on the biological sample with HER-2/neu and topolla specific probes.

In other embodiments, the instructions for determining comprises instructions for performing in-situ hybridization on the biological sample with a topolla specific probe, and instructions for performing immunohistochemical methods on the biological sample with anti-HER2 antibodies.

The present invention provides methods for diagnosing and treating cancer, and in particular methods for determining the susceptibility of subjects suspected of having breast cancer to treatment with topoisomerase II inhibitors. The present invention also provides in situ hybridization probes and kits for specifically detecting topolla gene sequences.

In some embodiments, the present invention provides methods for identifying a candidate for topoisomerase II inhibitor treatment, comprising: a) providing a candidate subject suspected of having cancer cells; b) detecting a copy number for both HER-2/neu and topolla in the cancer cells; and c) identifying the candidate subject as being suitable for treatment with a topoisomerase II inhibitor, wherein the identifying comprises demonstrating amplification of the copy number for both HER-2/neu and topolla. In some embodiments, the candidate subject has cancer cells. In other embodiments, the candidate subject has been previously diagnosed as having cancer cells from diseases including, but not limited to, leukemia, brain cancer, kidney cancer, lymphoma, eye cancer, connective tissue cancer, Hodgkin's disease, bone cancer, testicular cancer, cervical cancer, thyroid cancer, melanoma, skin cancer, uterine cancer, lung cancer, colon cancer, rectal cancer, ovarian cancer, bladder cancer, larynx cancer, prostate cancer, stomach cancer, breast cancer, and pancreatic cancer. In preferred embodiments, the candidate subject has breast cancer cells. In particularly preferred embodiments, the candidate subject has metastatic breast cancer cells.

The present invention provides methods for identifying candidates for topoisomerase II inhibitor treatment, comprising: a) providing a candidate subject suspected of having breast cancer cells; b) detecting a copy number for both HER-2/neu and topolla in the breast cancer cells; and c) identifying the candidate subject as suitable for treatment with a topoisomerase II inhibitor, wherein the identifying comprises demonstrating amplification of the copy number for both HER-2/neu and topolla. In certain embodiments, the demonstrating comprises comparing the copy number of both HER-2/neu and topolla to a control copy number. In further embodiments, the copy number of HER-2/neu is at least 1.5 times greater than the control copy number. In additional embodiments, the copy number of topolla is at least 1.5 times greater than the control copy number. In further embodiments, the method further comprises step d) treating the candidate subject with a topoisomerase II inhibitor.

In some particularly preferred embodiments, the candidate subject is a human. In other embodiments, the candidate subject is a non-human animal. In some embodiments, the animal is a mammal (e.g., human, cat, dog, pig, or cow). In some preferred embodiments, the animal is a female, in other embodiments, the animal is a male. In some embodiments, the candidate subject has breast cancer cells (e.g., previously diagnosed as having breast cancer cells). In some preferred embodiments, the breast cancer cells are metastatic.

In some embodiments of the present invention, the detecting step comprises obtaining a tissue sample (e.g., biopsy) comprising the breast cancer cells from the candidate subject. In further embodiments, the detecting step further comprises contacting the tissue sample comprising the breast cancer cells with a first probe specific for the HER-2/neu and a second probe specific for the topolla. In certain embodiments, the second probe comprises at least about 100,000 nucleotides (e.g. a probe library comprising 100,000 nucleotides) and hybridizes to a target region of human chromosome seventeen under in situ hybridization conditions, and wherein the target region contains topolla gene sequence, but does not contain HER-2/neu gene sequence.

In other embodiments, the first and second probes are detectably labeled nucleic acid. In further embodiments, the first probe is nucleic acid capable of hybridizing to HER-2/neu. In additional embodiments, the second probe is nucleic acid capable of hybridizing to topolla. In further embodiments, the first and second probes are detectably labeled. In particular embodiments, the detecting step comprises fluorescent in situ hybridization. In some embodiments, the detecting step comprises Southern blotting (hybridization) or Northern blotting (hybridization). In additional embodiments, the detecting step comprises Western blotting. In further embodiments, the detecting step comprises enzyme immunoassay (EIA). In certain embodiments, the detecting step comprises enzyme-linked immunosorbent assay (ELISA). In certain embodiments, the first and/or second probe is labeled with digoxigenin, and the first and/or second probe is fluorescently labeled. In other embodiments, the first and/or second probe is detected by chromogenic in situ hybridization. In certain embodiments, the first and/or second probe is detected by fluorescent in situ hybridization. In further embodiments, the detecting step comprises contacting the tissue sample comprising the breast cancer cells with an antibody specific for HER2 (e.g., in order to detect a copy number for HER-2/neu) and a nucleic acid probe specific for topolla. In some particularly preferred embodiments, the detecting step comprises immunohistochemical detection and fluorescent in situ hybridization (FISH). However, it should be noted that any suitable method for detection of topolla and HER-2/neu finds use with the present invention.

The present invention further provides methods for identifying candidates for topoisomerase II inhibitor treatment, comprising: a) providing a candidate subject suspected of having breast cancer cells; b) detecting a copy number for both HER-2/neu and topolla in the breast cancer cells, wherein the detecting comprises contacting the breast cancer cells with a first probe specific for HER-2/neu, a second probe specific for topolla (e.g. a topolla probe library comprising fragments), and a control probe; and c) identifying the candidate subject as being suitable for treatment with a topoisomerase II inhibitor, wherein the identifying comprises demonstrating amplification of the copy number for both HER-2/neu and the topolla. In particular embodiments, the control probe is specific for human chromosome 17. In some particularly preferred embodiments, the topoisomerase II inhibitor is an anthracycline. In other embodiments, the anthracycline is selected from doxorubicin and epirubicin. In further embodiments, the breast cancer cells are metastatic.

The present invention provides methods for identifying candidates for topoisomerase II inhibitor treatment, comprising: a) providing a candidate subject comprising breast cancer cells, wherein the breast cancer cells comprise an amplified copy number for HER-2/neu, b) detecting a copy number topolla in the breast cancer cells; and c) identifying the candidate subject as suitable for treatment with a topoisomerase II inhibitor, wherein the identifying comprises demonstrating amplification of the copy number for topolla. In particular embodiments, the demonstrating comprises comparing the copy number for topolla to a control copy number. In further embodiments, the copy number of the topolla is at least 1.5 times greater than the control copy number. In certain embodiments, the candidate subject is known to have an amplified copy number for HER-2/neu (e.g., previously determined by immunohistochemistry, FISH, chromogenic in situ hybridization, CISH, ELISA, etc.).

The present invention further provides methods comprising; a) providing a subject with cancer, wherein the subject comprises cancer cells with an amplified copy number of HER-2/neu and topolla, and b) treating the subject with a topoisomerase II inhibitor. In other embodiments, the candidate subject has been previously diagnosed as having cancer cells from diseases including, but not limited to, leukemia, brain cancer, kidney cancer, lymphoma, eye cancer, connective tissue cancer, Hodgkin's disease, bone cancer, testicular cancer, cervical cancer, thyroid cancer, melanoma, skin cancer, uterine cancer, lung cancer, colon cancer, rectal cancer, ovarian cancer, bladder cancer, larynx cancer, prostate cancer, stomach cancer, breast cancer, and pancreatic cancer. In preferred embodiments, the candidate subject has breast cancer cells. In particularly preferred embodiments, the candidate subject has metastatic breast cancer cells.

The present invention also provides methods comprising: a) providing a subject with breast cancer, wherein the subject comprises breast cancer cells with an amplified copy number of HER-2/neu and topolla, and b) treating the subject with a topoisomerase II inhibitor. In some embodiments, the topoisomerase II inhibitor is an anthracycline. In particular embodiments, the anthracycline is selected from doxorubicin and epirubicin. In further embodiments, the breast cancer cells are metastatic. In particularly preferred embodiments, the subject is a human. In other embodiments, the subject is a non-human animal. In still further embodiments, the animal is a mammal (e.g., human, cat, dog, pig, and cow). In preferred embodiments, the animal is a female, while in other embodiments, the animal is a male.

The present invention also provides compositions comprising a probe, the probe comprising at least about 100,000 nucleotides, wherein the probe hybridizes to a target region of human chromosome seventeen under in-situ hybridization conditions, and wherein the target region contains topolla gene sequence, but does not contain HER-2/neu gene sequence. In preferred embodiments, the probe comprises a library of fragments ranging in size from about 0.1 kb to about 15 kb, preferably about 0.3 kb about 10 kb, and more preferably about 0.5 to about 4 kb. In certain embodiments, the probe comprises a library of fragments that hybridize to a region about 170 kb in size (e.g. 100 kb to 250 kb) containing the topolla gene, but does not contain the HER2 gene sequence.

In certain embodiments, the probe comprises no more than 1 million nucleotides. In other embodiments, the probe comprises no more than 500,000 nucleotides, while in other embodiments, the probe comprises no more than 250,000 nucleotides. In further embodiments, the probe comprises about 140,00 to 200,000 nucleotides (e.g. as a probe library of fragments). In preferred embodiments, the probe comprises about 170,000 nucleotides. In particular embodiments, the probe comprises at least about 125,000, 140,000, 150,000, or 160,000 nucleotides. In some embodiments, the probe contains less than ten, less than five, or less three percent repetitive nucleic acid sequences (e.g., ALU and LINE elements). In other embodiments, the probe contains less than two percent, or less than 1 percent repetitive nucleic acid sequences.

In particular embodiments, the probe further comprises a label. In certain embodiments, the label comprises digoxigenin. In other embodiments, the label is florescent. In particular embodiments, the label comprises biotin.

In certain embodiments, the target region is at least about 500,000 nucleotides from the HER-2/neu gene sequence (e.g. the site where the probe hybridizes on human chromosome 17 is at least 500,000 bases away from the HER2/neu gene). In other embodiments, the target region is at least about 400,000 or 300,000 or 200,000 nucleotides from the HER2/neu gene. In some preferred embodiments, the probe does not falsely detect HER2/neu instead of topolla. Also in some preferred embodiments, the target region target region comprises human chromosome locus 17q11-21.

In certain embodiments, the present invention provides kits and systems comprising the probe described above and at least one additional component. In some embodiments, the kits and systems of the present invention comprise; a) a composition comprising a probe (e.g. a library of fragments ranging in size from about 0.1 kb to about 10 kb), the probe comprising at least about 100,000 nucleotides, wherein the probe hybridizes to a target region of human chromosome seventeen under in-situ hybridization conditions, and wherein the target region contains topolla gene sequence, but does not contain HER-2/neu gene sequence, and b) at least one other component (e.g. insert component, primary antibody, secondary antibody, HER2 or HER2/neu probe, one or more buffers, digestion solution, cover slips, slides, graded alcohols, SSC buffer, etc). Examples 10 and 11 provide additional components for inclusion in the kits of the present invention.

In some embodiments, the insert component comprises written material. In certain embodiments, the written material comprises instructions for using the probe (e.g. in an ISH procedure such as FISH or CISH). In other embodiments, the written material comprises instructions for testing patient breast cancer tissue samples to determine if a patient should be treated with a topoisomerase II inhibitor or an anti-HER2 antibody.

In certain embodiments, the probe further comprises a label (as detailed above). In some embodiments, the kits and systems of the present invention further comprise a first antibody specific for the label (e.g., FITC-anti-digoxigenin antibody). In particular embodiments, the kits and systems of the present invention further comprise a second antibody specific for the first antibody (e.g., HRP-anti-FITC antibody).

In other embodiments, the kits and systems of the present invention further comprise a second probe, wherein the second probe specifically detects HER2 or HER2/neu. In preferred embodiments, the second probe does not falsely detect topolla.

### DESCRIPTION OF THE FIGURES

FIG. 1 shows the results of immunohistochemical and fluorescent in situ hybridization detection in 34 primary breast cancer samples.

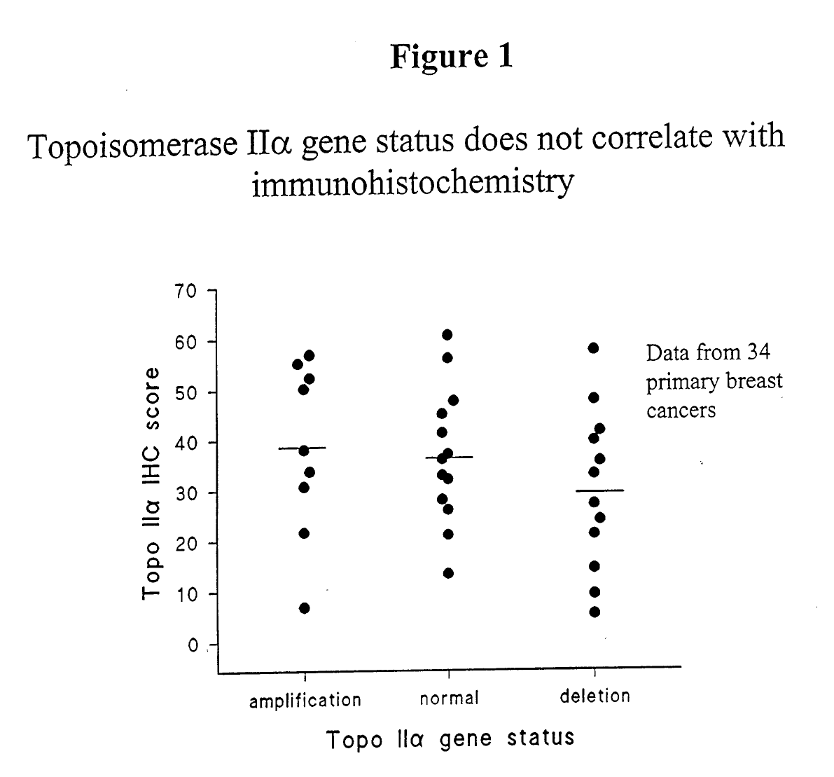

FIG. 2 shows the 3′ end of the Exemplary topolla probe (SEQ ID NO:9), and the 5′ end of the Exemplary topolla probe (SEQ ID NO:10).

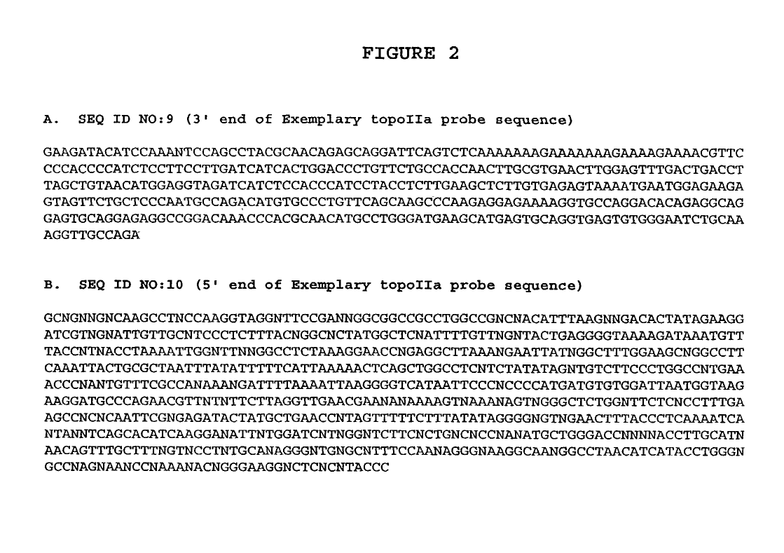

FIG. 3 shows chart useful for interpreting ISH results using topolla and chromosome 17 probes.

FIG. 4 shows the BAC clones used in Example 14 that flank the ABL gene.

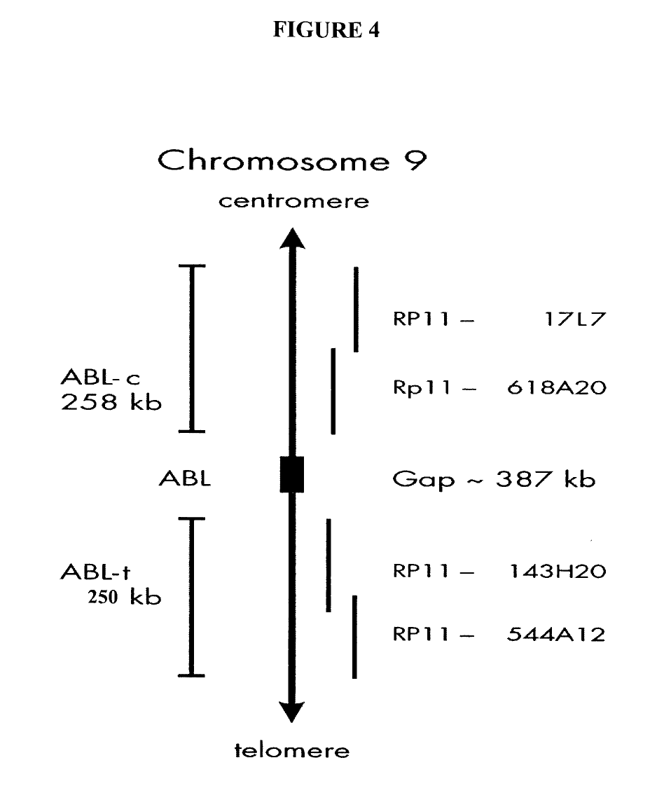

FIG. 5 shows ABL translocations, partner genes involved and Leukemias with ABL translocations.

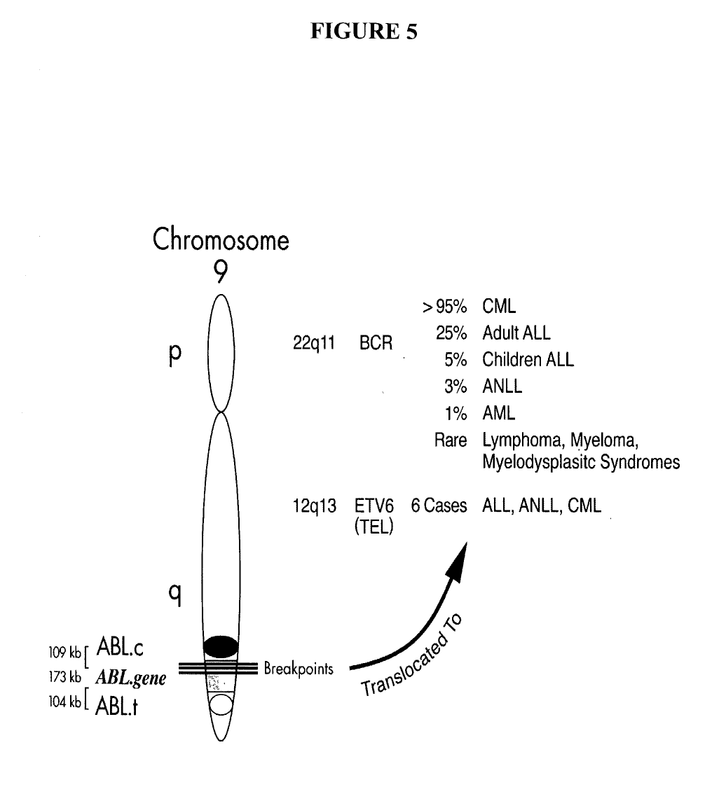

FIG. 6A shows a schematic diagram of ABL DNA, and FIG. 6B shows various breakpoints in the ABL gene.

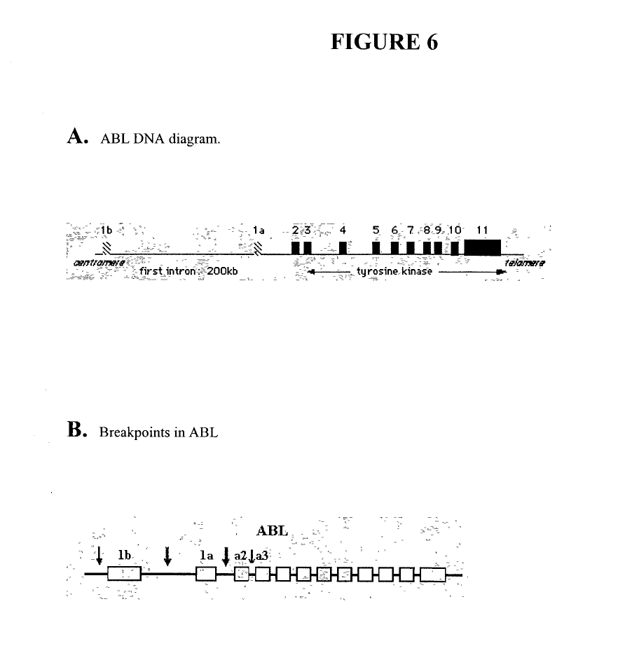

FIG. 7 shows BCR-ABL translocations.

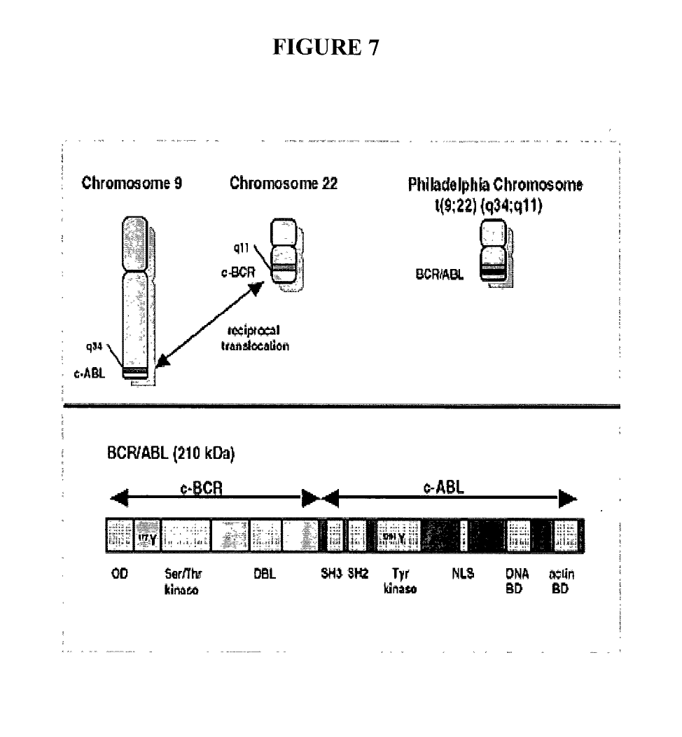

FIG. 8 shows simplified scheme of the BCR and ABL genes with indicated breakpoints, along with exemplary BCR/ABL transcripts and proteins originating from individual breaks on the BCR and ABL genes.

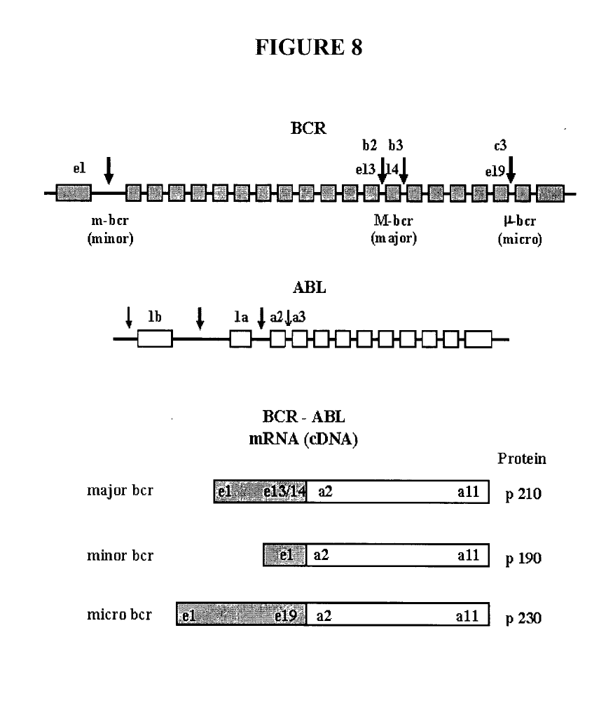

FIG. 9 shows clinicopathogic correlates of the most common BCR-ABL fusions.

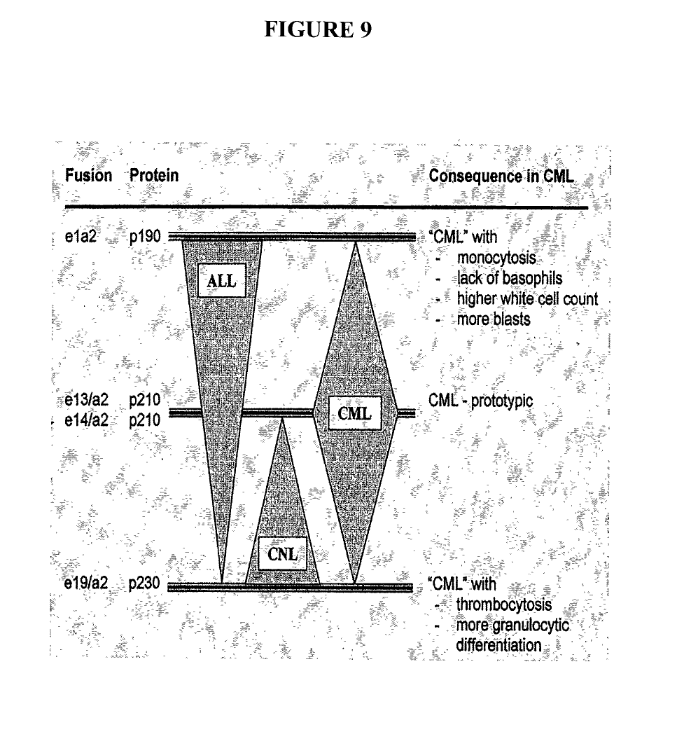

FIG. 10 shows UCSC genome browser for ABL gene.

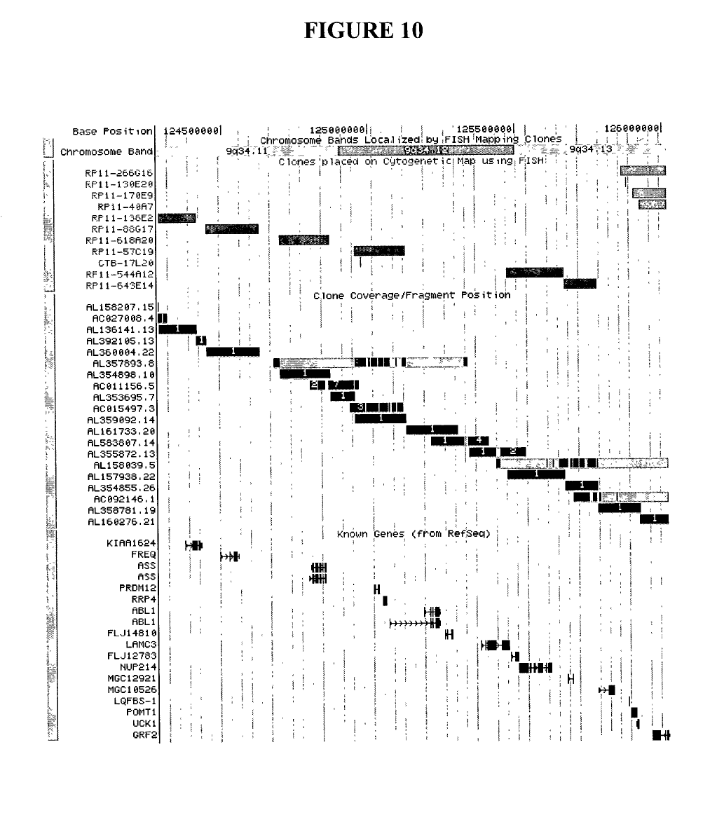

FIG. 11 shows a schematic illustration of ABL translocation detection by dual-color in situ hybridization (e.g. CISH or FISH). Black dots represent ABL.c and white dots represent ABL.t. Partial karyotyptes and the corresponding interphase nuclei are shown in the figure. Normal cells without ABL translocations show black and white dots in juxtaposition, while cells with ABL translocation show one pair of black and white dots separated. Cells with ABL translocation and deletion of chromosomal material centromeric to the ABL gene breakpoint show one pair of black and white dots in juxtaposition and the black dot in another pair is disappeared (deleted).

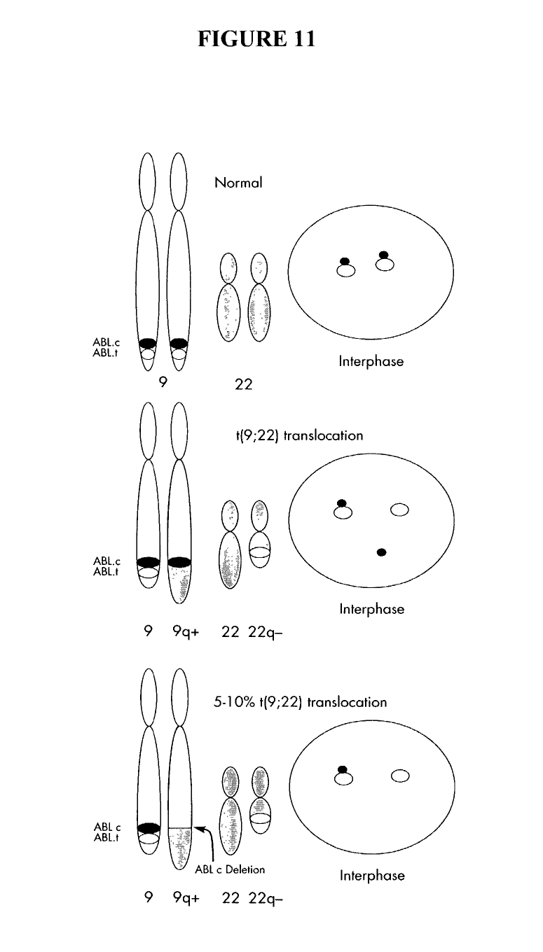

### DEFINITIONS

To facilitate an understanding of the present invention, a number of terms and phrases are defined below:

As used herein, the term “candidate subject”, “subject” or “patient” refers to an animal like a dog, cat, bird, livestock, and preferably a human. In some embodiments, the subject is suspected of having cancer that may be evaluated for suitability for topoisomerase II inhibitor treatment or anti-HER2 immunotherapy. Examples of subject and candidate subjects include, but are not limited to, human women suspected of having breast cancer and human men suspected of having breast cancer.

As used herein, the term “copy number” as used in reference to specific nucleic acid sequences (e.g., HER-2/neu, topolla and control) refers to the actual number of these sequences per single cell. Copy number may be reported for one single cell, or reported as the average number in a group of cells (e.g., tissue sample). When comparing the “copy number” of cells (e.g., experimental and control cells) one need not determine the exact copy number of the cell, but instead need only obtain an approximation that allows one to determine whether a given cell contains more or less of the nucleic acid sequence as compared to another cell. Thus, any method capable of reliably directly or indirectly determining amounts of nucleic acid may be used as a measure of copy number even if the actual copy number is not determined.

As used herein, the term “HER-2/neu” refers to a nucleic acid sequence encoding the HER2 protein, and includes both the wild-type sequence and naturally occurring variations, truncations, and mutations.

As used herein, the term “topolla” refers to a nucleic acid sequence encoding Topolla protein, or portions thereof, and includes both the wild-type sequence and naturally occurring variations, truncations, and mutations.

As used herein, the term “suitable for treatment with topoisomerase II inhibitors” when used in reference to a candidate subject refers to subjects who are more likely to benefit from treatment with topoisomersase II inhibitors than a subject selected randomly from the population. For example, using the screening methods of the present invention as described in Example 6, 79% of the subjects selected responded to topoisomerase II inhibitor treatment (as compared to 10% or less if subjects were randomly selected from the population, or as compared to approximately 30-40% of metastatic breast cancer patients).

As used herein, the term “amplification” when used in reference to copy number refers to the condition in which the copy number of a nucleic acid sequence (e.g., HER-2/neu) is greater than the copy number of a control sequence (e.g., chromosome 17). In other words, amplification indicates that the ratio of a particular nucleic acid sequence (e.g., HER-2/neu) is greater than 1:1 when compared to a control sequence (e.g., 1.1:1, 1.2:1, or 1.3:1). In preferred embodiments, the ratio of a particular nucleic acid sequence is at least 1.5 times greater than the control sequence copy number (i.e., 1.5:1).

As used herein, the term “nucleic acid molecule” and “nucleic acid sequence” refer to any nucleic acid containing molecule including, but not limited to DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “probe” refers to an oligonucleotide (i.e., a sequence of nucleotides), or a library of nucleotide fragments, whether occurring naturally as in a purified restriction digest or produced synthetically, recombinantly or by amplification (e.g. PCR), which is capable of hybridizing to an oligonucleotide of interest. Probes useful in the present invention may be single-stranded or double-stranded. Probes are useful in the detection, identification and isolation of particular gene sequences (e.g., HER-2/neu, topolla, and chromosome 17). It is contemplated that any probe used in the present invention may be labeled with any “reporter molecule,” so that is detectable in any detection system, including, but not limited to enzyme (e.g., ELISA, as well as enzyme-based immuno-histochemical assays), fluorescent (e.g., FISH), radioactive, mass spectroscopy, and luminescent systems. It is not intended that the present invention be limited to any particular detection system or label.

As used herein, the term “label” refers to any molecule which may be detected. For example, labels include, but are not limited to, ³²P, ¹⁴C, ¹²⁵I, ³H, ³⁵S, biotin, digoxigenin, avidin, fluorescent or enzymatic molecules.

As used herein, the phrase “repetitive nucleic acid sequences” refers to nucleic acid sequence within a genome which encompass a series of nucleotides which are repeated many times, often in tandem arrays. The repetitive sequences can occur in the genome in multiple copies ranging from two to hundreds of thousands of copies and may be clustered or interspersed on one or more chromosomes throughout a genome. Although repetitive nucleic acid sequences may be present throughout the genome, a large number of the repetitive nucleic acid sequences are typically located at the centromere of each chromosome. Examples of repetitive nucleic acid sequences include, but are not limited to, ALU and LINE elements.

As used herein, the terms “in situ hybridization” and “ISH” refer to methods for detecting and localizing nucleic acids within a cell or tissue preparation. These methods provide both quantitative and spatial information concerning the nucleic acid sequences within an individual cell or chromosome. ISH has been commonly used in many areas, including prenatal genetic disorder diagnosis, molecular cytogenetics, to detect gene expression and overexpression, to identify sites of gene expression, to map genes, to localize target genes and to identify various viral and microbial infections, tumor diagnosis, in vitro fertilization analysis, analysis of bone marrow transplantation and chromosome analysis. The technique generally involves the use of labeled nucleic acid probes which are hybridized to a chromosome or mRNA in cells that are mounted on a surface (e.g slides or other material). The probes can be labeled with fluorescent molecules or other labels. One example of fluorescent in situ hybridization (FISH) is provided in Kuo et al., Am. J. Hum. Genet., 49:112-119, 1991 (hereby incorporated by reference). Other ISH and FISH detection methods are provided in U.S. Pat. No., 5,750,340 to Kim et al., hereby incorporated by reference. Further examples of fluorescent in situ hybridization, as well as chromogenic in situ hybridization are provided in Examples 1-10 below. Additional protocols are known to those of skill in the art.

As used herein, the phrase “under in situ hybridization conditions” refers to any set of conditions used for performing in situ hybridization (ISH) that allows the successful detection of labeled oligonucleotide probes. Generally, the conditions used for in situ hybridization involve the fixation of tissue or other biological sample onto a surface, prehybridization treatment to increase the accessibility of target nucleic acid sequences in the sample (and to reduce non-specific binding), hybridization of the labeled nucleic acid probes to the target nucleic acid, post-hybridization washes to remove unbound probe, and detection of the hybridized probes. Each of these steps is well known in the art and has been performed under many different experimental conditions. Again, examples of such in situ hybridization conditions are provided in Kuo et al., U.S. Pat. No. 5,750,340, and Examples 1-10 (below). Further examples of conditions and reagents useful for performing in situ hybridization are provided below.

The tissue or biological sample can be fixed to a surface using fixatives. Preferred fixatives cause fixation of the cellular constituents through a precipitating action which is reversible, maintains a cellular morphology with the nucleic acid in the appropriate cellular location, and does not interfere with nucleic acid hybridization. Examples of fixatives include, but are not limited to, formaldehyde, alcohols, salt solutions, mercuric chloride, sodium chloride, sodium sulfate, potassium dichromate, potassium phosphate, ammonium bromide, calcium chloride, sodium acetate, lithium chloride, cesium acetate, calcium or magnesium acetate, potassium nitrate, potassium dichromate, sodium chromate, potassium iodide, sodium iodate, sodium thiosulfate, picric acid, acetic acid, sodium hydroxide, acetones, chloroform glycerin, and thymol.

After being fixed on a surface, the samples are treated to remove proteins and other cellular material which may cause nonspecific background binding. Agents which remove protein include, but are not limited to, enzymes such as pronase and proteinase K, or mild acids, such as 0.02.-0.2 HCl, as well as RNase (to remove RNA).

DNA on the surface may then denatured so that the oligonucleotide probes can bind to give a signal. Denaturation can be accomplished, for example, by varying the pH, increasing temperature, or with organic solvents such as formamide. The labeled probe may then hybridize with the denatured DNA under standard hybridization conditions. The tissue or biological sample may be deposited on a solid surface using standard techniques such as sectioning of tissues or smearing or cytocentrifugation of single cell suspensions. Examples of solid surfaces include, but are not limited to, glass, nitrocellulose, adhesive tape, nylon, or GENE SCREEN PLUS.

As used herein, the term “polymerase chain reaction” (“PCR”) refers to the method described in U.S. Pat. Nos. 4,683,195, 4,683,202, and 4,965,188, hereby incorporated by reference, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²p-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the phrase “anti-HER2 antibody-free topoisomerase II inhibitor treatment” refers to a treatment regimen for a subject that includes administering topoisomerase II inhibitors (e.g. anthracyclines), but does not include anti-HER2 antibody administration at about the same time.

As used herein, the phrase “topoisomerase II inhibitor-free anti-HER2 antibody treatment” refers to a treatment regimen for a subject that includes the administration of anti-HER2 antibodies (e.g. HERCEPTIN), but does not include topoisomerase II inhibitor (e.g. anthracyclines) administration at about the same time.

As used herein, the phrase “subtracted probe library” refers to a mixture of nucleic acid fragments configured to hybridize to a target region (e.g. selected portion of a chromosome containing gene of interest) that comprises at least about 90 percent repeat free fragments.

### DESCRIPTION OF THE INVENTION

The present invention relates to chromogenic (calorimetric) in situ hybridization (CISH) and nucleic acid probes useful for in situ hybridization. Specifically, the present invention provides methods, kits, and compositions for performing bright-field cancer diagnostics employing chromogenic in situ hybridization (e.g. to detect gene amplifications, gene translocations, and chromosome polysomy). In preferred embodiments, the present invention provides CISH methods, kits and compositions for detecting HER2 gene status. The description of the invention is presented below in the following sections: I. Chromogenic In-Situ Hybridization; II. CISH HER-2/neu Detection and Anti-HER2 Antibody Therapy; III. Combined HER21HER-2/neu and topolla detection; IV. Combined CISH and IHC; V. Subtracted Probes; and VI. ABL Probe Pairs and Detecting BCR-ABL Translocations.

I. Chromogenic In Situ Hybridization

Chromogenic in situ hybridization (CISH) is a technique that allows in situ hybridization methods to be performed and detected with a bright-field microscope, instead of a fluorescence microscope as required for FISH. While FISH requires a modern and expensive fluorescence microscopes equipped with high-quality 60× or 100× oil immersion objectives and multi-band-pass fluorescence filters (not used in most routine diagnostic laboratories), CISH allows detection with standard light (bright-field) microscopes (which are generally used in diagnostic laboratories). Also, with FISH, the fluorescence signals can fade within several weeks, and the hybridization results are typically recorded with an expensive CCD camera, while the results of CISH do not generally fade allowing the tissue samples to be archived and reviewed later. Therefore, analysis and recording of FISH data is expensive and time consuming. Most importantly, tissue section morphology is not optimal in FISH on FFPE. Generally, histological detail is better appreciated with bright-field detection, which is possible with CISH detection. A further advantage of CISH is that large regions of tissue section can be scanned rapidly after CISH counterstaining since morphological detail is readily apparent using low power objectives (e.g. 10× and 20×), while FISH detection generally requires substantially higher magnification (thus reducing the field of view). These advantages generally make CISH a superior in situ hybridization technique compared to FISH.

General chromogenic/colorimetric in situ hybridization methods are described in WO0026415 to Fletcher et al. (herein incorporated by reference for all purposes). Particular reagents and steps for performing CISH on formalin-fixed, paraffin-embedded (FFPE) tissue samples, as well as cell sample/metaphase chromosome samples are described in WO0026415 and the section presented below. Importantly the description detailed below provides exemplary CISH methods, procedures, and reagents, and is not to be construed as limiting the present invention.

A. Formalin-Fixed, Paraffin-Embedded (FFPE) Tissue Samples

Generally, FFPE tissue samples (e.g. cancer biopsy tissue samples) will measure about 1-2 cm in diameter, but any type of diameter may be employed. This tissue sections (e.g. 4-5 um) may be mounted on treated (e.g. HISTOGRIP treated) microscope slides or other solid support surface (e.g. Superfrost/Plus microscope slides).

i. Pretreatment

In preferred embodiments, the FFPE tissue samples are first subjected to a deparaffinization step. This may be accomplished, for example, by exposing the sample to Xylene for about 10 minutes at room temperature. This may be repeated if necessary. The sample may then be exposed to EtOH (e.g. 100% EtOH) for about 5 minutes at room temperature. In preferred embodiments, this is performed three times. The tissue samples are then allowed to dry (e.g. air dry).

Next, tissue samples are subjected to a heat pretreament step. Specifically, a pretreatment buffer is added to the tissue samples, and the samples are heated to approximately 92-100 degrees Celsius for approximately 15 minutes (although varying incubation times may be used depending on the tissue fixation). Examples of pretreatment buffers included, but are not limited to, Citrate buffer, EDTA-TRIS buffer (e.g. 0.1 M Tris/0.05 M EDTA, pH 7.0), and TRIS buffer. In certain embodiments, the preheat temperature is achieved with a microwave, a pressure cooker, a hot plate, or other type of heating device. Also, in preferred embodiments, the preheat temperature is such that the pretreatment buffer boils. For example, a preferred temperature range is 96-100 degrees Celsius. A particularly preferred temperature range is 98-100 degrees Celsius. It was determined that the temperature range of 98-100 gives enhanced CISH detection results (e.g. as compared to 92 degrees Celsius). The tissue samples are then generally washed (e.g. with water or PBS) two or three times (e.g. for 2-4 minutes per wash).

Generally, the next step is an enzyme digestion step. In preferred embodiments, the tissue samples are exposed to pepsin digestion (e.g. at room temperature or at about 37° C.) for about a several minutes (e.g. 1-20 minutes may be required depending on tissue fixation). Importantly, excessive digestion may cause loss of nuclei and chromosome structure, while inadequate digestion may result in loss of signal. The tissue samples are then washed again (e.g. with water or PBS) two or three times (e.g. for 2-4 minutes per wash).

After washing, the tissue samples are then dehydrated with graded alcohols. For example, the tissue samples may be exposed to 70%, 85%, 95%, and 100% ethanol for about 2 minutes each time, and then air dried.

ii. Denaturation and Hybridization

Denaturation and hybridization may accomplished as one step (co-denaturing and hybridization, described in this paragraph), or as two steps (separate denaturation and hybridization, described below). One general procedure for co-denaturation and hybridization is as follows. First, add the probe (e.g. 12-20 ul of a subtracted probe library) to the center of a cover slip (e.g. 22×22 mm coverslip, or 24×32 mm coverslip, or coverslips described in WO0138848 to Ventana Medical Systems Inc., herein incorporated by reference). In other embodiments, the probe is added directly to the tissue sample. In other embodiments, the liquid COVERSLIP from Ventana Medical Systems, Inc. is applied over the tissue sample (e.g. to create a humid reaction chamber on the slide). In other embodiments, the Zymed CISH UNDERCOVER slips are employed (available from Zymed Labs.). In some embodiments, the coverslip is then placed probe side down on the tissue sample. The edges of the coverslip may then be scaled, for example, with a thin layer of rubber cement to prevent evaporation during incubation. The slide with the tissue sample is then placed on a slide block of PCR machine or on a heating block with temperature display (or other heating device). Denaturation is conducted at approximately 94-95 degrees Celsius for about 5-10 minutes. The tissue sample (e.g. on the slide) is then incubated at approximately 37 degrees Celsius for about 16-24 hours. Incubation may be conducted, for example, in a dark humidity box (or other humidified chamber) or in the slide block of a PCR thermal cycler.

One general procedure for separate denaturation and hybridization is as follows. This procedures is useful, for example, when a PCR machine or heating block are not readily available. First, the tissue sample is denatured in denaturing buffer (e.g. 4 ml 20× SSC [20× SSC buffer=0.3M Sodium Citrate, with 3M NaCl, ph 7.0], 8 ml ddH₂O, 28 ml formamide) at about 75 degrees Celsius for about 5 minutes. Increases in temperature may be used for additional samples being denatured at the same time (e.g. add about 1 degree Celsius for each additional sample being denatured). Next, the slides are denatured with graded alcohols (e.g. 70% EtOH, 85% EtOH and 95% all for about 2 minutes at negative 20 degrees Celsius, and then 100% EtOH for about 2 minutes twice).

Then the tissue samples are air dried, while the labeled probe (e.g. subtracted probe) is denatured at about 75 degrees Celsius for about 5 minutes. The denatured probe is then placed on ice. About 12-15 ul of the denatured probe is added to the center of a coverslip (e.g. a 22×22 mm coverslip, or other cover). The coverslip is then added to the appropriate tissue sample area, and the tissue sample is placed in a dark humid box (or other humidified chamber) at about 37° C. for at least about 14 hours. Next step, for example, would be the stringency wash below.

B. Cell Sample or Metaphase Chromosome Sample

i. Pretreatment

Initially, slides may be immersed in a pretreament buffer such as 2× SSC buffer (20× SSC buffer=0.3M Sodium Citrate, with 3M NaCl, ph 7.0), or Tris-EDTA, or Tris, at about 37 degrees Celsius for about 60 minutes. In some embodiments, the cell samples are treated with pepsin compositions (e.g. Zymed's SPOT LIGHT Cell Pretreatment Reagent) for about 5 minutes at about 37 degrees Celsius. Incubation time may be, for example, from about 1-10 minutes depending on cell type and slide-making conditions. Excessive pepsin digestion may cause loss of nuclei and chromosome structure. Inadequate digestion may result in loss of signal. Slides may then be washed (e.g. in dH₂O or PBS) for two or three time, for two or three minutes each time at room temperature. In some embodiments, the slides may be immersed in buffered formalin (e.g. 10%) for about a minute at room temperature. The slides may then be washed (e.g. in dH₂O or PBS) two or three times for about 1-3 minutes each time, at room temperature. The slides may then be dehydrated. For example, the slides may be dehydrated in 70%, 85%, 95%, and 100% ethanol for 2 minutes each, and then air dried. Slides may proceed to ISH procedures described below or stored (e.g. in 70% ethanol at −20 degrees Celsius).

ii. Denaturation and Hybridization

First, add the probe (e.g. 12-20 ul of a subtracted probe library, See Subtracted Probe section below) to the center of a cover slip (e.g. 22×22 mm coverslip, or 24×32 mm coverslip, or coverslips described in WO0138848 to Ventana Medical Systems Inc., herein incorporated by reference). In other embodiments, the probe is added directly to the tissue sample. In some embodiments, the liquid COVERSLIP from Ventana Medical Systems, Inc. is applied over the tissue sample (e.g. to create a humid reaction chamber on the slide). In other embodiments, the Zymed CISH UNDERCOVER slips are employed (available from Zymed Labs.). In some embodiments, the coverslip is then placed probe side down on the tissue sample. The edges of the coverslip may then be sealed, for example, with a thin layer of rubber cement to prevent evaporation during incubation. For denaturation, the slide with the tissue sample is then placed on a slide block of PCR machine or on a heating block with temperature display (or other heating device). Denaturation is conducted at approximately 80 degrees Celsius for about 2-5 minutes. The slides may then be placed in a dark humidity box (or other humidity chamber) or in the slide block of a PCR thermal cycler for about 16-24 hours at about 37 degrees Celsius.

iii. Stringency Wash

The remaining steps (e.g. stringency wash, immunodetection, counterstaining/ coverslipping) are generally the same for both cell sample and FFPE. After hybridization, the rubber cement (or other sealant used, if a sealant is used) and cover slip (or other cover) is carefully removed. The tissue sample slides are then washed (e.g. in Coplin jar) in order to remove unhybridized probes. For example, the tissue sample slides may be washed in 0.5× SSC at 72° C. for about 5 minutes. The temperature may be adjusted up if more than one slide is being washed (e.g. add 1° C. per slide for more than 2 slides, but preferable no higher than 80° C. The slides are then washed again in, for example, dH₂O or PBS/Tween 20 buffer for about 2-3 minutes. This may be repeated two or three times.

iv. Immunodetection

Generally, depending on the detection reagents used, the first step in preparation for immunodetection is peroxidase quenching and endogenous biotin blocking. For peroxidase quenching, slides may be submerged in 3% H₂O₂ in absolute methanol (e.g. add part 30% hydrogen peroxide to 9 parts absolute methanol) for about 10 minutes. The slide is then washed with PBS (e.g. 1×PBS (10 mM)/Tween 20 (0.025%)) for 2-3 minutes. This may be repeated two or three times. The tissue samples are then blocked. Blocking can be performed by adding 2 drops per slide (at room temperature) of CAS-BLOCK (which is 0.25% casein, 0.2% gelatin, and 10 mM PBS, pH 7.4). After about 10 minutes, the blocking reagent is blotted off.

Next, the labeled probe library is detected. The probe may be detected by first adding an anti-label primary antibody (e.g. a mouse antibody or antibody with a label such as FITC). In certain preferred embodiments, the probe is labeled with digoxigenin, and the primary antibody is an FITC-anti-dig antibody. In other preferred embodiments, the primary antibody is unlabelled, but is from a particular species such as rat, mouse or goat. In other embodiments, the primary antibody is linked (e.g. conjugated) to an enzyme (e.g. horseradish peroxidase (HRP) or alkaline phosphatase (AP)) able to act on a chromogenic substrate, and does not require the secondary antibody described below. Generally, about two drops of the primary antibody solution is added to the tissue at room temperature for about 30-60 minutes. The tissue sample is then rinsed, for example, with PBS (e.g., 1×PBS/Tween 20 (0.025%) for about 2-3 minutes. This may be repeated two to three times.

In preferred embodiments, a secondary antibody is added to the tissue sample that is able to bind to the primary antibody. For example, if the primary antibody is labeled with FITC, the secondary antibody may be an anti-FITC antibody. Also for example, if the primary antibody is an unlabeled mouse antibody, the secondary antibody may be an anti-mouse antibody (e.g. goat anti-mouse antibody). Generally, the secondary antibody is linked (e.g. conjugated) to an enzyme (e.g. HRP or AP) able to act upon a chromogenic substrate (or chemiluminescent substrate). Generally, about 2 drops of the secondary antibody is added to the tissue sample at room temperature for about 30-60 minutes. The tissue sample is then rinsed, for example, with PBS (e.g., 1×PBS/Tween 20 (0.025%) for about 2-3 minutes. This may be repeated two to three times. Additional antibodies (e.g. tertiary, quaternary antibodies) may be used if desired.

In certain preferred embodiments, the secondary antibody is linked to a polymer that is itself linked to many enzyme molecules (e.g. polymerized HRP or polymerized AP). This allows each individual antibody to connect (via the polymer) to many enzyme molecules in order to increase signal intensity. Such polymerized enzymes are known in the art, and are commercially available from, for example, Nichirei Inc. (Tokyo, Japan) and ImmunoVision.

Once the antibody (or other detection molecule) which is linked to an enzyme (e.g. a secondary or tertiary antibody conjugated to AP or HRP), is added to the biological sample, a substrate for the enzyme is then added. In preferred embodiments, the substrate is a chromogen. Examples of suitable chromogens include, but are not limited to, DAB, FAST RED, AEC, BCIP/NBT, BCIP/INT, TMB, APPurple, ULTRABLUE, TMBlue, and VEGA RED. In other embodiments, the substrate is a chemiluminescent molecule (e.g. BOLD APS 540 chemiluminescent substrate, BOLD APS 450 chemiluminescent substrate, or BOLD APB chemiluminescent substrate, all commercially available from INTERGEN Co.). Therefore, the next step, for example in developing the slide, is to mix DAB (or other substrate), buffer, and hydrogen peroxide (e.g. 0.6%) in a tube, then to add 3 drops per slide to the tissue sample for about 30 minutes. In certain embodiments, chromogen enhancers are added to increase signal intensity (e.g. AEC enhancer, FAST RED enhancer, and DAB enhancer available from INNOVEX Biosciences, ZYMED Labs, etc.). The tissue sample may then be washed (e.g. with running tap water) for about two minutes. In certain embodiments, the immunohistochemistry steps are automated or partially automated. For example, the ZYMED ST 5050 Automated Immunostainer may be employed to automate this process.

V. Counterstaining and Coverslipping

In some embodiments, the next step is a counterstaining and coverslipping step. This step may be performed by counterstaining the tissue sample. For example, the tissue sample may be counterstained with hematoxylin or other counterstain. This procedure may be performed for about 6 seconds to about 1 minutes, depending on the type of tissue being stained. Preferably, overly dark counterstaining is avoided so as not to obscure the positive signal. The slides may then be washed (e.g. with running tap water) for a couple of minutes, and then, in some embodiments, dehydrated with graded EtOH (e.g. 70%, 85%, 95%, 100%, 100% for about 2 minutes each, repeated two times). In some embodiments, the dehydration is not performed with EtOH, when, for example, FAST RED is the substrate (e.g. a water soluble substrate). The slides may then be exposed to Xylene for about two minutes (this may be repeated at least once). The tissue sample may then be coverslippped (e.g. with HISTOMOUNT, Cytoseal 6.0, cat. # 8310-16, Stephen Scientific). In some embodiments, CLEARMOUNT is employed instead (e.g. when FAST RED is one of the substrates).

vi. Microscopy and Interpretation of Results

Importantly, the slides may be visualized using standard bright-field microscopy using a bright-field microscope (e.g. OLYMPUS, NIKON, LEITZ, etc.). Generally, probes are visible with about 20× magnification (e.g. 15×-25×). In preferred embodiments, probes are visualized with about 30×, or 40× (e.g. 28×-43×) magnification. Higher powers (e.g. 60×, 80×, and 100×) may be employed, but are generally not necessary (and may reduce the field of view). In some embodiments, for evaluating translocation results, a 100× oil lens is employed. In other embodiments, for evaluating amplification and centromere probes, 40× lens is employed. Below are examples of how CISH results may be interpreted for gene amplification/centromere detection, as well as for gene translocation.

As mentioned above CISH detection of gene amplification, translocation, and cetromere detection may be performed with a bright-field microscope, or other type of microscope. For example, in general, CISH staining results are clearly seen using a 40× objective in tissue sections which are counterstained (e.g., hematoxylin). An individual gene or chromosome centromere signal normally appears as a small, single dot. Targeted gene amplification is typically seen as large chromogen-stained (e.g. DAB-stained) clusters or many dots in the nucleus or mixed clusters and multiple dots (e.g., ≧6 dots per nucleus). Tumors with no targeted gene amplification typically show 1 to 5 dots per nucleus. Normally, 3-5 dots per nucleus in more than 50% of tumor cells are due to chromosome polysomy. Table 1 shows an exemplary chart useful for CISH visualization of individual genes for chromosome polysomy.

TABLE 1
Exemplary CISH Signal Visualization for an individual gene
or chromosome centromere

| Magnification | CISH Signal |
|---|---|
| 10× | Individual signals are barely visible and may be |
| | missed. |
| 20× | Individual signals are small but clearly discernible. |
| 40× | Individual signals are easily identified. |
| 60× or 100× | Not Necessary |

Examples of CISH detection and interpretation of gene amplification in HER2 and Topolla CISH, are presented in Tables 2 and 3 below.

TABLE 2
Exemplary Assessment of HER2 gene status by CISH

| | |
|---|---|
| Amplification | >10 copies or large clusters of HER2 gene |
| | (amplicon) per nucleus in >50% of cancer cells. |
| Low Amplification | 6-10 copies of HER2 gene or small cluster of HER2 |
| | gene (amplicon) per nucleus in >50% of cancer |
| | cells. |
| | Labeled chromosome 17 centromere probe may be |
| | applied for CISH to confirm that 6-10 copies of |
| | HER2 gene (<5% cases) were due to HER2 gene |
| | amplification but not chromosome 17 polysomy. |
| No Amplification | 1-5 copies of HER2 gene per nucleus in >50% of |
| | cancer cells. |
| | 3-5 copies of HER2 gene per nucleus is due to |
| | chromosome 17 polysomy. There is no need for |
| | chromosome 17 centromere CISH. |
| | Occasionally, it is found that HER2 has 3-5 copies |
| | and chr.17cen has 1-2 copies in >50% of cancer |
| | cells (HER2/chr.17cen ratio is ≧2), it is due to what |
| | sometimes was seen by CGH of duplication of |
| | chromosome arm 17q. |

TABLE 3
Exemplary Topo IIα Probe and Chromosome 17 Centromeric
Probe Usage

| Topo IIα | | Chromosome 17 Centromeric |
|---|---|---|
| Status | Topo IIα Results | Probe |
| Deletion | When Topo IIα gene copy number is less than the | |
| | centromeric copy number. | |
| Normal diploid | 2 copies | 2 copies |
| Aneuploidy | 3-5 copies | 3-5 copies |
| Amplification | Gene cluster (amplicon) | Gene amplification is highly |
| | or ≧6 separate copies | likely, Chromosome 17 |
| | | Centromeric Probe analysis is |
| | | not necessary |

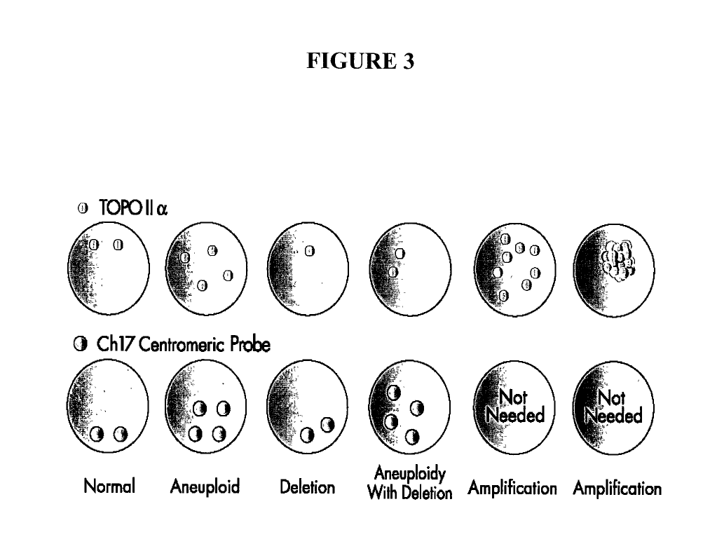

Also, in some normal cells, one gene copy may be missing due to loss of nuclear material during sectioning. Therefore, in general, analysis should be based on the results from the majority of cancer cells (>50%) observed. FIG. 3 presents one interpretation chart for interpreting topolla amplification using topolla and chromosome 17 centromere probes. It should be noted that these are representative examples only. Copy numbers from actual samples may vary for aneuploidy, deletion, and amplification.

We claim:

1. A method for performing chromogenic in-situ hybridization, comprising;
a) preheating a biological sample in a pretreatment buffer at a temperature of at least 96 degrees Celsius,
b) exposing said biological sample to a enzyme digestion solution,
c) contacting said biological sample with a subtracted probe library under conditions such that said subtracted probe library hybridizes to a target region in said biological sample,
d) adding a detection molecule linked to an enzyme to said biological sample under conditions such that said detection molecule binds; i) to said labeled subtracted probe library, or ii) an intermediate molecule linked to said subtracted probe library, and
e) adding a colorimetric substrate to said biological sample.

2. The method of claim 1, further comprising step f) detecting said target region.

3. The method of claim 2, wherein said detecting comprising visualizing said colorimetric substrate with a microscope.

4. The method of claim 3, wherein said microscope is a bright-field microscope.

5. The method of claim 1, wherein said subtracted probe library is configured for detecting HER2 gene amplification.

6. The method of claim 1, wherein said subtracted probe library is configured for detecting topolla gene amplification.

7. The method of claim 1, wherein said subtracted probe library is configured for detecting EGFR gene amplification.

8. The method of claim 1, wherein said subtracted probe library is configured for detecting N-MYC gene amplification.

9. The method of claim 1, wherein said subtracted probe library comprises a probe pair library.

10. The method of claim 9, wherein said probe pair comprises a split-apart probe pair.

11. The method of claim 9, wherein said probe pair library comprises; i) a first probe library configured to hybridize to a first region of chromosome nine that is centromeric with respect to the ABL gene, and ii) a second probe library configured to hybridize to a second region of chromosome nine that is teleomeric with respect to the ABL gene.

12. The method of claim 9, wherein said probe pair library comprises; i) a first probe library configured to hybridize to a first region of chromosome eighteen that is centromeric with respect to the SYT gene, and ii) a second probe library configured to hybridize to a second region of chromosome eighteen that is teleomeric with respect to the SYT gene.

13. The method of claim 1, wherein said temperature is at least 98 degrees Celsius.

14. The method of claim 1, wherein said temperature is from 96 degrees Celsius to 100 degrees Celsius.

15. The method of claim 1, wherein said subtracted probe library is about 90 percent free of repeat sequences.

16. The method of claim 1, wherein said subtracted probe library is about 95 percent free of repeat sequences.

17. A kit for performing chromogenic in-situ hybridization, comprising;
a) a labeled subtracted probe library, wherein said subtracted probe library is configured to hybridize to a target region,
b) a written insert component, wherein said written inert component comprises instructions for performing chromogenic in-situ hybridization.

18. The kit of claim 17, further comprising at least one of the following; pretreatment buffer, an enzyme digestion solution, a colorimetric substrate, and a detection molecule conjugated to a calorimetric substrate enzyme.

19. The kit of claim 17, wherein said instructions for performing chromogenic in-situ hybridization comprises instructions for visualizing said calorimetric substrate with a bright-field microscope.

20. The kit of claim 17, wherein said subtracted probe library is configured for detecting HER2 gene amplification.

21. The kit of claim 17, wherein said subtracted probe library is configured for detecting topolla gene amplification.

22. The kit of claim 17, wherein said subtracted probe library is configured for detecting EGFR gene amplification.

23. The kit of claim 17, wherein said subtracted probe library is configured for detecting N-MYC gene amplification.

24. The kit of claim 17, wherein said subtracted probe library comprises a probe pair library.

25. The kit of claim 24, wherein said probe pair comprises a split-apart probe pair.

26. The kit of claim 24, wherein said probe pair library comprises; i) a first probe library configured to hybridize to a first region of chromosome nine that is centromeric with respect to the ABL gene, and ii) a second probe library configured to hybridize to a second region of chromosome nine that is teleomeric with respect to the ABL gene.

27. The kit of claim 24, wherein said probe pair library comprises; i) a first probe library configured to hybridize to a first region of chromosome eighteen that is centromeric with respect to the SYT gene, and ii) a second probe library configured to hybridize to a second region of chromosome eighteen that is teleomeric with respect to the SYT gene.

28. The kit of claim 17, wherein said written insert component comprises instructions for preheating a biological sample in a pretreament buffer to a temperature of at least 96 degrees Celsius.

29. The kit of claim 17, wherein said written insert component comprises instructions for preheating a biological sample in a pretreament buffer to a temperature of at least 98 degrees Celsius.

30. The kit of claim 17, wherein said subtracted probe library is about 90 percent free of repeat sequences.

31. The kit of claim 17, wherein said subtracted probe library is about 95 percent free of repeat sequences.

32. A method for diagnosing and treating a subject, comprising;
a) preheating a biological sample from a subject in a pretreatment buffer,
b) exposing said biological sample to a enzyme digestion solution,
c) contacting said biological sample with a subtracted probe library under conditions such that said subtracted probe library hybridizes to a target region in said biological sample, wherein said target region comprises the HER2 gene sequence,
d) adding a detection molecule linked to an enzyme to said biological sample under conditions such that said detection molecule binds; i) to said labeled subtracted probe library, or ii) an intermediate molecule linked to said subtracted probe library,
e) adding a colorimetric substrate to said biological sample,
f) detecting said target region by visualizing said colorimetric substrate with a bright-field microscope, thereby determining that said biological sample has amplification of said HER2 gene sequence, and
g) identifying said subject as suitable for treatment with anti-HER antibodies.

33. The method of claim 32, further comprising step h) administering said anti-HER2 antibodies to said subject.

34. The method of claim 32, wherein said anti-HER2 antibodies comprise HERCEPTIN.
